(19) United States
(12) Patent Application Publication
(10) Pub. No.: US 2019/0011436 A1
(43) Pub. Date: Jan. 10, 2019

(54) A METHOD FOR IN SITU DETECTION OF BREASTMILK SPOILAGE

(71) Applicant: KONINKLIJKE PHILIPS N.V., Eindhoven (NL)

(72) Inventors: KIRAN HAMILTON J. DELLIMORE, EINDHOVEN (NL); SUZANNE MAAIKE VALSTER, EINDHOVEN (NL); NICOLAAS PETRUS WILLARD, EINDHOVEN (NL); DOORTJE VAN DE WOUW, EINDHOVEN (NL)

(21) Appl. No.: 16/079,409

(22) PCT Filed: Feb. 6, 2017
(86) PCT No.: PCT/EP2017/052512

§ 371 Date: Aug. 23, 2018

(30) Foreign Application Priority Data
Feb. 23, 2016 (EP) 16156851.4

(51) Int. Cl. G01N 33/52; A61J 9/00; G01N 33/04

(52) U.S. Cl. CPC G01N 33/526; A61J 2200/70; G01N 33/04; A61J 9/00

(57) ABSTRACT

The invention provides amongst others kit of parts (1000) comprising a container (200) comprising a container enclosure (210), the container enclosure (210) comprising a container enclosure internal face (211), and a container closure (220), the container closure (220) comprising a container closure internal face (221), wherein one or more of the container enclosure internal face (211) and the container closure internal face (221) comprises a CO2 detector (100), and wherein one or more of the container enclosure (210) and the container closure (220) comprise a light transmissive part (233) for external visual inspection of the CO2 detector (100).

### FIELD OF THE INVENTION

The invention relates to a detector for evaluation of expressed breast milk quality (or quality of other (liquid) food products, or other features of (liquid) food products). The invention further relates also to a method for evaluating expressed breast milk quality (or quality of other (liquid) food products).

### BACKGROUND OF THE INVENTION

Breast milk screening methods are known in the art. US2013/0040289, for instance, describes a test kit for detecting the presence of a target analyte in a fluid sample. In particular, the test kit includes reagents capable of detecting the target analyte of interest in breast milk. More particularly, the test kit is capable of detecting the presence of alcohol, caffeine, nicotine, drugs of abuse, therapeutic drugs, triglycerides, lactose, capsaicin, and gluten, for example, in breast milk. This documents, amongst others, describes a method of protecting a nursing infant from exposure to a target analyte present in breast milk that may have adverse effects on the nursing infant, said method comprising the steps of: (a) obtaining a breast milk sample from a subject, wherein the subject is suspected of ingesting a target analyte that may be harmful to the nursing infant; (b) detecting the presence, absence, or concentration of the target analyte in the breast milk by detecting a change in at least one reagent; and (c) if the change indicates the presence of the target analyte or concentration of the target analyte above a predetermined level, waiting a period of time to allow for clearance of the substance from the breast milk prior to breast feeding.

WO2001/04624 describes a colorimetric device which includes a silicone oligomer or polymer. The device is characterized in that the silicon oligomer or polymer is an organic solvent soluble, substantially non-curable, hydrophobic silicone oligomer or polymer. The colorimetric device comprises a pH sensitive dye. Further, the colorimetric device is characterized in that the silicone oligomer or polymer has an oxygen permeability of up to 150,000 cm per 24 hrs.

Michael Lu et al., “Milk Spoilage: Methods and Practices of Detecting Milk Quality”, Food and Nutrition Sciences, vol. 04, no. 07, 1 Jul. 2013, describes that milk spoilage is an indefinite term and difficult to measure with accuracy. This uncertainty can cause suffering for both milk manufacturers and consumers. Consumers who have been misled by ambiguous expiration dates on milk cartons waste resources by disposing of unspoiled milk or experience discomfort from drinking spoiled milk. Consumers are often unwilling to purchase products close to their inaccurate expiration dates. This consumer behavior has a negative financial impact on milk producers. Inaccurate milk spoilage detection methods also force milk producers to use overly conservative expiration dates in an effort to avoid the legal and economic consequences of consumers experiencing ill-ness from drinking spoiled milk. Over the last decade, new methods have been researched with the purpose of developing more accurate and efficient means of detecting milk spoilage. These methods include indicators based on pH bacteria counts and gas-sensor arrays. Michael Lu et al. explore various methods of spoilage detection designed to prevent such consequences. The respective level of effectiveness of each method is discussed in this paper, as well as several further approaches to contain freshness regardless of detection.

### SUMMARY OF THE INVENTION

Many mothers express breast milk into bottles, cups or pouches in order to store it for later consumption by their baby (e.g., if a mother has to return to work and cannot always be present to feed her baby or to enable a partner to feed the baby so that the mother can sleep uninterrupted during the night), to maintain or boost their breast milk supply (e.g., if a mother must temporarily stop nursing due to administration of medication that can be harmful to the baby) or to relieve pain and pressure from engorged breasts. In most cases, expressed breast milk is often not consumed directly or within a couple of hours. Fresh breast milk can be stored at room temperature (up to 25° C.) for only about 6-8 hours and therefore, generally needs to be refrigerated at 4° C. (safe up to 5 days) or frozen (safe from 2 weeks to 3 months depending upon the freezing temperature) for later use. After storing the milk longer than advised, bacterial growth may have spoiled the milk such that the milk is not safe anymore to give to the baby.

Many mothers, especially in Africa (where it is very hot and humid, travel distances are long, access to refrigeration is limited and electricity is unreliable), are often unsure whether their expressed and stored (at room temperature or refrigerated) breast milk is still safe for consumption by their baby. A similar problem arises for mothers of formula fed babies, who also may store the prepared formula milk for an extended period of time, such as in the case of night feeding and in the case of left over breast/formula milk (after the feeding). The same is also true for those working mothers who need to transport formula or breast milk during travel to and from their job.

At the moment many mothers assess stored breast milk by only subjective measures like the appearance or smell. But often these might be misleading as separation, discoloration, and smell changes are common. Many mothers do not want to taste the milk, but also do not want to throw away the milk if not needed because it took them a lot of time and effort to collect it. There is a huge need and opportunity for a simple, reliable and cheap solution to measure spoilage objectively for them.

The pH of fresh, healthy (nutritious) and safe breast milk typically ranges between 7.0 and 7.4. Changes in fresh breast milk pH occur during the postpartum lactation period when the milk is pumped from the mother's breast. In general, the pH of milk remains between 7.0 and 7.1 until three months postpartum and then increases gradually to 7.4 by 10 months postpartum. Moreover, over time, after being pumped from the breast and stored, the pH of breast milk appears to decrease (i.e., become more acidic) by at least 2 units over a 24 hour time period in the temperature range between 15° C. and 38° C. At 15° C. human breast milk is safe for 24 hours while at 25° C. it is safe for only 4 hours. The pH of stored milk changes over time due to a combination of proteolysis (i.e., protein breakdown into smaller polypeptides or amino acids) and lipolysis (lipid breakdown into glycerol and free fatty acids). The formation of fatty and amino acids leads to a decrease in breast milk pH over time from neutral to acidic pH levels (pH <7.0). Both lipolysis and proteolysis are linked to the growth of certain bacteria in the milk which secrete lipase and protease enzymes to digest (i.e., breakdown) amino acids, fatty acids and proteins (e.g., Staphylococcus aureus, Streptococcus viridians and Staphylococcus albus, etc. A byproduct of this breakdown (i.e., fermentation) process is the release of carbon dioxide, which can be correlated with the change in pH and thus spoilage of the milk.

Currently, there are no guidelines for mothers to determine whether expressed breast milk is still safe for consumption by her baby. It is advised to smell and taste the milk before feeding it to the baby. However, due to freezing and thawing, the color, smell and structure (i.e., appearance) of the milk can change significantly. This leads to uncertainty on the mother's part and may result in the feeding of spoiled milk to the baby or the wastage of unspoiled milk.

Other more sophisticated methods for detecting milk spoilage exist. These include using pH sensitive litmus paper, an electronic pH meter or biochemical analysis. However, all of these approaches have significant drawbacks. Litmus paper is low cost and disposable, but it is cumbersome to use and can be difficult to interpret especially in the narrow pH range over which breast milk is safe for consumption. Also it can contaminate the milk by leaching of the pH indicator and it is not sterile. A pH meter and its probes are expensive, it requires electrical power as well as burdensome storage/maintenance conditions (e.g., electrodes must be stored in acidic conditions), and it is cumbersome for use in milk testing. Other chemical analysis techniques using assays or Raman spectrometry exist however they are only suitable in laboratory environments and are impractical for at home use. One may also use an assay, but this may also be expensive and complicated to use.

Furthermore, it might be possible to develop a sticker which changes color depending on the breast milk temperature or formula milk temperature. The sticker, which may expire at the same rate as the milk spoils, can be placed on a bottle of pumped milk and depending on the temperature and time, a color (change) may indicate whether in general the milk would be spoiled. However, this sticker does not directly measure whether the milk is spoiled or not (the bottle might for instance not have been cleaned well enough). Nor does it measure the degree of degradation or the nutritional value.

Hence, it is an aspect of the invention to provide an alternative solution to detect breast milk quality, which preferably further at least partly obviates one or more of the above-described drawbacks, and which especially allows a direct and timely measure of the quality, but without affecting the expressed breast milk, and which solution is relatively easy and/or cost effective.

In a first aspect, the invention provides a CO₂ detector (“detector”), especially for application in a closed space enclosing a liquid food (and having a head space), wherein the CO₂ detector comprises a detector enclosure containing a pH indicator, wherein the detector enclosure is impermeable to liquid water, wherein at least a first part of the detector enclosure comprises a membrane, especially a silicone membrane, that is permeable for CO₂.

It surprisingly appeared that the CO₂ concentration is indicative of the quality of expressed breast milk, and is an earlier warning signal than the pH of the milk. It appears that when breast milk starts to deteriorate the pH starts to drop hours after the start of the deterioration, whereas the CO₂ concentration is already increasing. Hence, a CO₂ indicator is therefore more safe and direct than a pH indicator for the breast milk (or formula milk or optionally another type of (liquid) food product). Further, a pH indicator of the breast milk implies expensive electrodes or contact of the breast milk with such indicator.

However, the proposed solution allows a contactless, in situ analysis of milk pH to detect spoilage (such as due to proteolysis and lipolysis of the milk). Further, the proposed solution overcomes the issue of leaching of potentially harmful chemical substances (i.e., pH indicator, reagents, reactants and products) used for milk analysis into the breast or formula milk. Yet further, the present solution may overcome the issue of uncertainty over the safety of expressed breast milk or formula milk (or other milk, such as cow milk, goat milk, etc.) caused by the variable rate of degradation of breast and formula milk over time due to variability in storage conditions. The present solution may be relatively cheap, simple and safe, which can easily be used by consumers at home. In embodiments, the invention provides a pH indicator on a carrier which is enclosed in a semi-porous (i.e., open for gasses and vapors but closed for liquids), especially hydrophobic, silicone shield. An immobilization (i.e., binding) of the pH indicator on the carrier may reduce the risk of leaching of the pH indicator into the milk, as does the hydrophobic silicone shield, which prevents contact between the pH indicator and the milk. In addition, the semi-porous nature of the silicone allows diffusion of carbon dioxide from the headspace (i.e., air) above the milk or within the milk (which may occur, e.g., when the container is transported in a bag) into the enclosure with the pH indicator, thereby enabling the in situ pH measurement, while also preventing large and/or charged molecules from passing out of the milk. The proposed approach is especially intended to be used for in situ analysis of breast or formula milk spoilage, such as in the form of a (single-use)(sterile) dipstick that is inserted directly into the milk after it is placed in a bottle, milk pouch or cup for feeding to the baby or a (reusable) sticker which is inserted into the lid or on the side of milk cup, milk pouch or baby bottle. Hence, the CO₂ indicator may in embodiments be integrated in the container enclosure or the closure thereof.

The invention provides a CO₂ detector. The detector is especially designed for application in a closed space (such as a container space). Such closed space, may enclose a food product, especially a liquid food, such as breast milk, or formula milk, or other type of milk. However, the food product may also comprise e.g. pesto, mayonnaise, etc. Further, the closed space may not entirely be occupied by the food product, but may also include free space, such as a headspace of a (liquid) food product. The detector may especially be configured within such space at a position where free space is expected (such as close to a closure). The CO₂ detector and its application is especially described in relation to CO₂ detection of milk. However, the CO₂ indicator may also be applied for other application such as the fermentation of a food product. The food product may in embodiments also include yoghurt or wine. The food product is especially liquid. The term liquid may in embodiments also refer to (pourable) semi-solid food products, such as a paste. A semi-solid food product may especially have the ability to flow under pressure. Especially, the semi-solid food product may be pourable.

The CO₂ detector comprises a detector enclosure which is impermeable to liquid water. The impermeability may be characterized by a water adsorption which is especially equal to or smaller than 0.1% w/w. Hence, in this way a (liquid) food product, such as breast milk cannot enter the detector enclosure. In this way, breast milk, or another (liquid) food product cannot be contaminated with material contained by the enclosure. The phrase “impermeable to liquid water” especially relates to ambient conditions, i.e. room temperature (20° C.) and 1 bar liquid water.

Especially, the enclosure is of a food grade material, such as one or more of high density polyethylene (HDPE), low density polyethylene (LDPE), polypropylene (PP), etc. Such materials may be substantially impermeable for liquid water or gas. Further, such materials may be substantially transmissive for light, allowing inspection of part of the space enclosed by the detector enclosure (i.e. the pH indicator (see below)).

However, at least part of the detector enclosure is permeable for CO₂ (but still not permeable for liquid water) (CO₂ especially refers to gaseous CO₂). Such permeable part allows introduction of CO₂ into the detector enclosure, by which the CO₂ indicator can indicate a concentration of CO₂, whereby the CO₂ indicator is e.g. indicative of the quality of the food product. Especially, at least a first part of the detector enclosure comprises a silicone membrane that is permeable for CO₂. Further, especially the detector enclosure is also not permeable to the pH indicator (dye), fats and proteins from milk. In this way, contamination of a food product, such as milk, and/or the sensor may be prevented.

The membrane is especially a silicone membrane. The silicone membrane thus comprises silicone. Note that the entire enclosure may be of silicone. The silicone is permeable for CO₂, but not for liquid water, indicator dye, fats, lipids, proteins, etc. The silicone membrane may e.g. comprise a silicone layer having a thickness in the range of 0.2-5 mm, such as 0.5-5 mm, such as 1-4 mm. By its nature, the silicone may have the non-permability for liquid water and the permeability for CO₂.

The permeability (of the membrane) for water vapor is especially at least 20,000 cm³ (STP) cm cm⁻² s⁻¹ cmHg₋₁, such as at least 40,000 cm³ (STP) cm cm⁻² s⁻¹ cmHg⁻¹. The permeability of CO₂, is especially at least 1,000 cm³ (STP) cm cm⁻² s⁻¹ cmHg⁻¹, such as especially at least 2000 cm³ (STP) cm cm⁻² s⁻¹ cmHg⁻¹, like at least 3000 cm³ (STP) cm cm⁻² s⁻¹ cmHg⁻¹. All reference values are provided at RT (20° C.) and ambient pressure, i.e. about 1 bar air pressure. The first part, such as the (silicone) membrane, may be hydrophobic. In this way, permeability of liquid water is also prevented. The detector enclosure, especially the first part, may however be permeable to water vapor (see also below). The pH indicator may be a (thin) layer of pH indicator material. The pH indicator may have an area of e.g. in the range of 1-1000 mm², like 4-400 mm², such as 4-200 mm². This pH indicator area may be visible to a user (through the light transmissive part).

In specific embodiments, the silicone membrane comprises polydimethyl siloxane, even more especially substantially consists of polydimethyl siloxane. Alternatively or additionally, the siloxane may comprise aryl groups, like phenyl. Other options may also be possible, but good results were obtained with the dimethyl silicone. In yet further specific embodiments the silicone membrane comprises a silicone rubber. Such rubber may especially be permeable for CO₂ (and H₂O vapor). Herein, the term “membrane” is used, as the material is substantially closed, like a closed layer, but nevertheless permeable to one or more species, such as at least CO₂.

The CO₂ detector is essentially based on a pH indicator. CO₂ together with water forms acids. Hence, the concentration of CO₂ in a (headspace of a (closed) container can be translated into a pH value indicated by the pH indicator. Hence, the detector enclosure especially contains a pH indicator. Therefore, basically the CO₂ detector comprises a pH indicator.

For a change in the pH also water may be necessary, as the pH indicator may especially be based on a pH change of water. Hence, in embodiments the silicone membrane is permeable to water vapor. In this way, CO₂ and water vapor in the headspace may penetrate into the detector enclosure and reach the pH indicator. Alternatively or additionally, in embodiments the detector enclosure also contains water. For instance, the indicator may bind water, or may be soaked with water.

In further embodiments, the detector enclosure may also contain a hygroscopic material, such as e.g. one or more selected from the group consisting of sodium carbonate, sodium chloride, silicon dioxide, etc. PVP, dextran, polyethyleen glycol (PEG), threhalose, sorbitol, glycerol etc. Such hygroscopic material may retain water that may be necessary for the pH indicator.

In specific embodiments, however, especially the pH indicator is not comprised in a matrix but is provided per se. In this way, the pH indicator may indicate relatively fast. As indicated herein, the pH indicated may be attached to a polymeric ion, but is especially essentially not mixed with such polymer. For instance, the pH indicated may be attached to a polymeric surface, such as a polymeric coating.

The volume of the detector enclosure may be relatively small, such as in the range of 0.1-10,000 mm³, especially 0.2-5,000 mm³. Hence, the water available in the enclosure may not be freely flowable, but may be bound or absorbed, such as especially bound or absorbed by the pH indicator. Hence, in embodiments the detector enclosure is a detector envelope.

The pH indicator is especially a halochromic chemical compound. Especially, the pH indicator is a chemical detector for hydronium ions (H₃O⁺)/hydrogen ions (H⁺). The indicator causes the color of a solution (here water available in the detector enclosure) to change depending on the pH. Examples of pH indicators are e.g. Methyl red-Bromocresol green (mixture), Bromocresol green, Chrysoidin, Alyzarin red S, Cochineal/carmine, Resazurin, 4-Phenylazo-l-naphthylamine, Ethyl red 2-(p-dimethylaminophenylazo)pyridine (mixture), Resorcin blue, 4-(p-ethoxyphenylazo)-m-(phenylene-diamine monohydrochloride, Propyl red, Bromocresol purple, Chlorophenol red, Bromothymol blue, Phenol red, thymol blue, malachite green, etc. etc. In embodiments, the pH indicator is especially configured to change color at a pH selected from the range of 4-8, such as especially 5-7.

In yet further embodiments, the pH indicator comprises a pH indicator ion bound to a polymeric ion. One can e.g. use polymeric electrolytes. pH indicators may e.g. comprise organic molecules with sulfonic acids. They, or other pH indicator ions, can form an insoluble complex with a positively charged polymeric electrolyte, like chitosan, poly-diallylamine, poly-diallyldimethylamine, amino-dextran poly ethylene imine, poly lysine, or other proteins with lysine, etc. In this way, the pH indicator may be bound, and thereby better be contained in the detector enclosure.

Hence, in embodiments the pH indicator is provided on a support, which is herein also indicated as “substrate” or “carrier”. The detector enclosure may be the support or at least part of the detector enclosure may be comprised by the support. In embodiments, the pH indicator may be immobilized on the support. Hence, in these ways the present device provides a pH indicator provided on a support. In embodiments, the support may be light transmissive. Hence, in embodiments the support may comprise the light transmissive part. In further embodiments, the support may comprise glass or a light transmissive polymeric material, such as HDPE, LDPE, or PP (see also above). in yet further embodiments, the support may (also) comprise a silicone. In embodiments, the entire enclosure (including support) may be silicone.

For protection of the silicone material, the silicone material, such as a silicone rubber, may also include additives such as e.g. UV light blockers. Hence, in embodiments the silicone membrane comprises one or more of a UV absorber material and a UV reflector material.

The pH indicator can be “regenerated” by exposing the pH indicator to the ambient. When exposed to the ambient, ambient CO₂ concentrations are experienced which will lead to a color different from the color at elevated CO₂ concentrations. In this way, the CO₂ detector can be used a plurality of times. However, in other embodiments the CO₂ detector may be a single-use only. In other embodiments, the detector can be used a plurality of times. After some time, such as a plurality of pH changes, the pH indicator may become less effective. Hence, in embodiments the CO₂ detector may further comprise a CO₂-detector end-of-life indicator, such as a second (food-safe) dye, which changes color (e.g. turns black) after a time period has elapsed, which indicates the detector is no longer reliable.

Silicones can be made light transmissive. Especially, the silicone membrane is light transmissive (i.e. transmissive for visible light), thereby allowing inspection of the pH indicator. However, alternatively or additionally, another part of the CO₂ detector may be light transmissive. Hence, in embodiments one or more of the first part and an optional second part comprises a light transmissive part for visual inspection of the pH indicator externally from the CO₂ detector. The light transmissive part may also be indicated as “window”. It especially comprises a piece of material that is at least partly transmissive for visible light.

The CO₂ detector may be used in different configurations. For instance, the CO₂ detector may be included in a container or in a container closure, in a stick for arranging in a container, comprised by a (reusable) sticker, etc. etc.

Hence, in a further aspect the invention provides a container comprising a container enclosure, with in embodiments the container enclosure comprising a container enclosure internal face, wherein the container enclosure internal face comprises the CO₂ detector as described herein. The container can be used in combination with a container closure, for closing an opening or inlet in the container. Examples of containers include bottles, cups or pouches, etc. The container enclosure, together with the container closure may provide a closed space, substantially not accessible to CO₂ and water vapor. In this way, milk, or another food product, may be stored in the closed container. The container closure may e.g. include the bottom and wall of a container, such as a bottle. Hence, the container closure is especially configured to close an opening in the container enclosure. Such opening may especially be configured to allow pouring contents out of the container enclosure or to introduce contents into the container enclosure. The container closure is especially configured to close an opening in the container enclosure in such a way, that introduction of CO₂ or O₂ is minimized, as known in the art. The term “closed container” especially refers to the combination of a container enclosure and container closure which are configured to each other to provide a closed container, containing the space. The term “container enclosure” may especially refer to the functional combination of a container wall and container bottom which are especially arranged as container with container opening. The container opening can be closed with the container closure.

In yet a further aspect, the invention provides a container closure, wherein the container closure comprises a container closure internal face, with in embodiments the container closure internal face comprising the CO₂ detector as described herein. The container closure can especially be used in combination with a container, for providing a closed container.

During use, at least part of the container enclosure internal face may be in contact with the food product, such as expressed breast milk or formula milk.

The (liquid) food product, such as expressed breast milk, can be introduced into the container space (via the opening or inlet). The CO₂ detector is comprised by the container, or by the container closure, or may be introduced in another way (see also below). Inspection of the CO₂ detector may be through the container enclosure and/or through the container closure, or inspection may be possible by opening the container and inspecting the CO₂ detector, optionally after removal from the container space.

Therefore, in embodiments one or more of the container enclosure and the container closure comprise a light transmissive part for external visual inspection of the CO₂ detector (when the container is closed with the container closure).

Especially, the container enclosure comprises glass or a light transmissive polymeric material, such as HDPE, LDPE, or PP. Further, especially the container closure comprises a light transmissive polymeric material, such as HDPE, LDPE, or PP, though this may not be necessary when the container enclosure comprises already comprises a light transmissive material (and vice versa).

In yet a further aspect, the invention provides a kit of parts comprising (a) a container comprising a container enclosure, the container enclosure comprising a container enclosure internal face, and (b) a container closure, the container closure comprising a container closure internal face, wherein one or more of the container enclosure internal face and the container closure internal face comprise the CO₂ detector according to any one of the preceding claims, and wherein one or more of (i) the container enclosure and (ii) the container closure comprise a light transmissive part for visual inspection of the CO₂ detector from external of the closed container (when the container is closed with the container closure). The kit of parts may especially provide a container enclosure and a container closure, which are designed for each other, i.e. the container closure is designed to close the container, such as with a turn closure. Especially, the container is configured to contain expressed breast milk and/or to contain formula milk (i.e. the mixture of the formula milk powder and water for consumption by a baby). Such containers are known in the art. However, the invention may also be used for other applications. Especially, the container closure and container enclosure are designed to provide in an easy (and intuitive way) a good closing of the container. For instance, a click closure may be applied. Alternatively or additionally, a (silicone) seal may be applied. Hence, one or more of the container enclosure and container closure may include a (silicone) seal. Optionally, in embodiments the seal may be part of the CO₂ detector.

In embodiments, the container closure internal face comprises said CO₂ detector. With such embodiment, contact with a liquid food product can be minimized, as the closure internal face may in general be in contact with the head space over the liquid food product.

In embodiments, the CO₂ detector is detachable associated with the container enclosure internal face or the container closure internal face. Also in this way the container enclosure internal face or the container closure internal face may comprise the CO₂ detector. This allows removal of the CO₂ detector before e.g. boiling the container after use for disinfection. For instance, a detachable sticker may comprise the CO₂ detector. Especially, such sticker, but more in general all materials comprised by the CO₂ detector (that may come into contact with a (liquid) food product), may essentially consist of food grade material. Hence, in a further aspect the invention also provides a reusable sticker comprising the CO₂ detector as defined herein.

Alternative to a (temporary) association of the CO₂ detector with a container enclosure or container closure, also the CO₂ detector may be configured temporary in the container. For instance, a stick with the CO₂ detector may be arranged in the container, whereafter the container may be closed. Especially, a positioning element (such as a stick or a disc) may have a length of at least 70% of an internal diagonal length of the container to be used, such as at least 80%. Especially, the CO₂ detector is configured at one end of the positioning element, such that positioning of the CO₂ detector in the headspace is facilitated. Hence, in a further aspect, the invention also provides an elongated positioning element, configured to position in a container, wherein the positioning element comprises the CO₂ detector as defined herein.

Next to freshness, one may also be interested to know the temperature of the milk or other food product in the container. This could be while storing/transporting, but mainly to check the temperature when feeding (after heating), such as in the case of milk. Hence, the container closure or the container enclosure may further comprise a temperature indicator, which is especially configured to indicate a temperature with a color indication. In embodiments, the temperature indicator and the CO₂ detector may be integrated in a single device.

The invention also provides a kit with a plurality of CO₂ detectors, for instance a kit with a container (enclosure) and a plurality of closures comprising CO₂ detectors, respectively. The invention may also provide a kit with the CO₂ detector and information how to use the CO₂ detector. Hence, in yet a further aspect, the invention also provides a kit of parts comprising (a) one or more items selected from the group consisting of the container enclosure as defined herein, the container closure as defined herein, the elongated positioning element as defined herein, and the reusable sticker as defined herein, wherein one or more items comprise the CO₂ detector as defined herein, and wherein the kit of parts especially further comprises (b) one or more of a manual or a link to a manual on the internet. Hence, the information may be comprised in a manual included in a box or other package, containing the container, printed on the container and/or on the container closure, printed on the box or other package, etc. etc. However, also a link may be provided, such as a QR code printed on the container and/or container closure, and/or on the box or other package, etc. etc. Such QR code may be read by a smartphone or i-Phone or other camera including device, which may contain an App for reading such link. Optionally, a picture of the CO₂ detector may be used to interpret (via an App and/or internet) the (color of the) pH indicator. The manual may include information about what color(s) indicate a safe or unsafe food product, how to apply the CO₂ detector, information about lifetime of the CO₂ detector, etc. etc. For instance, in embodiment the detector may be configured to indicate indications like e.g. ‘fresh’, ‘ok’ (watch out, will start spoiling), ‘spoiled’, or any other steps in between, or other suitable indications.

In yet a further aspect, the invention also provides a method of evaluating a food product, especially a liquid food product, such as milk, even more especially expressed breast milk, the method comprising inspecting a CO₂ detector as defined herein in a container or retrieved from the container, wherein the container contains the food product, especially the liquid food product, such as milk, even more especially the expressed breast milk, and wherein inspection is executed after the CO₂ detector has been contained in the container, especially in a headspace over said food product, especially the liquid food product, such as milk, even more especially the expressed breast milk, during a (predetermined) period wherein the container (i.e. the container enclosure) is closed with a container closure. Here, the phrase “inspecting a CO₂ detector” may thus especially imply checking the color of the pH indicator (or the aqueous liquid within the detector enclosure). Based thereon, the quality of the (liquid) food product may be determined. Hence, with this method, amongst others the quality of expressed breast milk may be evaluated. Hence, expressed breast milk or prepared formula milk stored in a container, can now relatively easily be evaluated on the suitability to feed to a baby. For instance, after a few days in the refrigerator, a person can inspect the CO₂ detector through the light transmissive part of the container or container closure, and determine whether or not the food product is still consumable, such as whether or not the expressed breast milk or prepared formula milk can be fed to a baby. For instance, the detector may be configured to indicate when the (liquid) food product, such as (expressed breast) milk is not safe to be consumed anymore. However, the invention is not limited to the evaluation of expressed breast milk (or prepared formula milk). The principle of the invention can also be used for other applications.

In yet a further aspect, the invention provides a CO₂ detector (100) for application in a closed space (1) enclosing a liquid food, wherein the CO₂ detector (100) comprises a detector enclosure (110) containing a pH indicator (120), wherein the detector enclosure (110) is impermeable to liquid water, wherein at least a first part (131) of the detector enclosure (110) comprises a membrane (140) that is permeable for CO₂, wherein the membrane has a membrane thickness selected from 900 μm*323.10⁹ cm³ (STP) cm cm⁻² s⁻¹ cmHg⁻¹/(P)+/−y %, wherein the thickness is larger than 0 μm, wherein P is the permeability in 10⁹ cm³ (STP) cm cm⁻² s⁻¹ cmHg⁻¹ of a material of the membrane. One or more of the first part (131) and an optional second part (132) of the detector enclosure (110) also comprises a light transmissive part (133) for external visual inspection of the pH indicator (120) of the CO₂ detector (100). Here, y is especially 50 (i.e. +/−50%), even more especially y is 40, like 30, even more especially 20. Further, the membrane thickness is especially at least 40 μm, such as at least 100 μm, such as e.g. up to about 1200 μm, like up to about 1000 μm, such as up to about 900 μm. Embodiments herein described also apply to this aspect, unless the contrary is clear from the context. Hence, the membrane thickness is especially selected from 900 μm*323.10⁹ cm³ (STP) cm cm⁻² s⁻¹ cmHg⁻¹/(P) +/−30%, wherein the thickness is larger than 0 μm, and wherein P is the permeability in 10⁹ cm³ (STP) cm cm⁻² s⁻¹ cmHg⁻¹of a material of the membrane. The value of 323.10⁹ cm²/g/(cm.Hg) is the permeability of silicone. The unit 10⁹ cm³ (STP) cm cm ⁻² s⁻¹ cmHg⁻¹ can also be indicated as 10⁹ cm³*cm/cm²*s*cmHg) or 10⁹ cm³ (STP)*cm/(cm²*s*cmHg).

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1b schematically depict some aspects of possible applications of the invention;

FIGS. 2a-2c schematically depict some embodiments of the CO₂ detector;

FIGS. 3a-3b schematically depict some embodiments of kits of parts; and

FIGS. 4a-4c show some results.

The schematic drawings are not necessarily on scale.

### DETAILED DESCRIPTION OF THE EMBODIMENTS

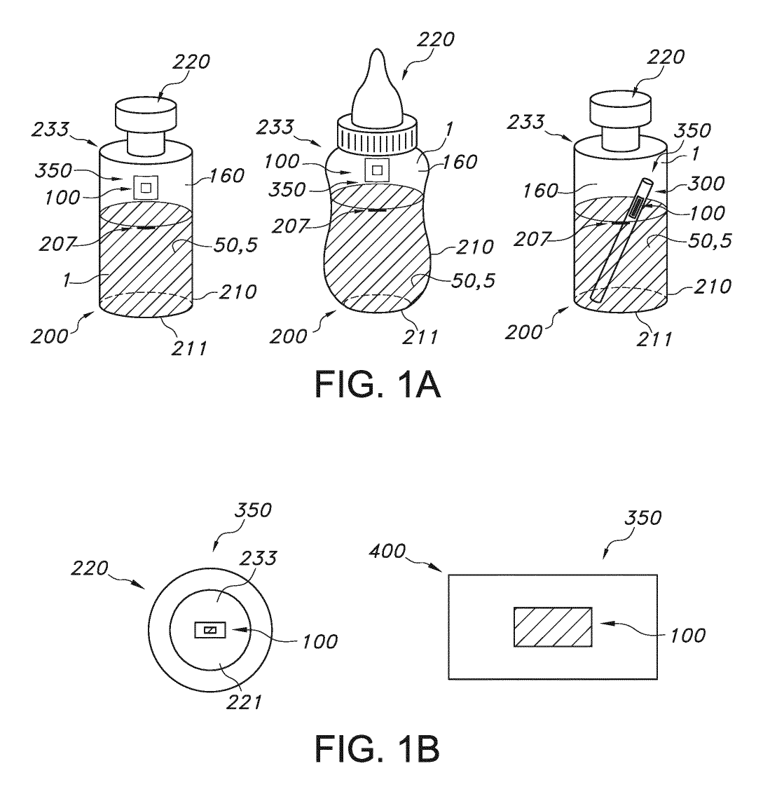

FIGS. 1a-1b schematically depict some aspects of possible applications of the invention. Here, a container 200 is depicted. The container 200 comprises a container enclosure 210. The container enclosure 210 comprises a container enclosure internal face 211, which at least partly encloses a container space or space 1. Further, a container closure 220 is depicted. The container enclosure 210 and the container closure 220 are especially configured for each other, such as to allow providing a closed container 200 (wherein a liquid can be stored without risk of escape from the liquid when the container is in the closed state).

The container closure 220 comprises a container closure internal face 221 (see FIG. 1b), with on the left a top view of a container closure 220 comprising the CO₂ detector 100 and on the right a sticker 400, such as a reusable sticker, comprising the CO₂ detector.

FIGS. 1a schematically depict some embodiments of the CO₂ detector 100, which is attached or integrated in the container enclosure (internal face) in the first two variants, or which is attached to or integrated into a separate elongated positioning element 300, which can be arranged in the container 200 (before closure of the container 200). Note that the separate elongated positioning element 300, here comprising a stick, has a length of at least about 70% of an internal diagonal length of the container to be used, such as at least 80%, but in general less than 100% of such diagonal length. Reference 350 indicates a substrate or carrier, herein also indicated as “support”, such as the elongated positioning element 300 or a sticker 400, etc.

One or more of the container enclosure internal face 211 and the container closure internal face 221 comprise the CO₂ detector 100. Especially, one or more of the container enclosure 210 and the container closure 220 comprise a light transmissive part 233 for external visual inspection of the CO₂ detector 100. FIG. 1b also schematically depicts a (reusable) sticker 400 comprising the CO₂ detector 100. Reference 160 indicates the headspace over the (liquid) food product, such as expressed breast milk. Reference 50 indicates a liquid or liquid food product; reference 5 indicates expressed breast milk, an example of the liquid food product 50. Referring to the embodiments schematically depicted in FIG. 1a, the CO₂ detector 100 can e.g. be integrated in the enclosure 210 or closure 220, or can e.g. be attached (as sticker 400) to the enclosure internal face 211 or container closure internal face 221.

FIG. 1a schematically depict three possible embodiments of containers 100, which comprise a container enclosure 210 and container closure 220, configured for each other to provide a closed container, with the container closure 220 configured to close an opening (not visible in the closed state) of the container enclosure 210.

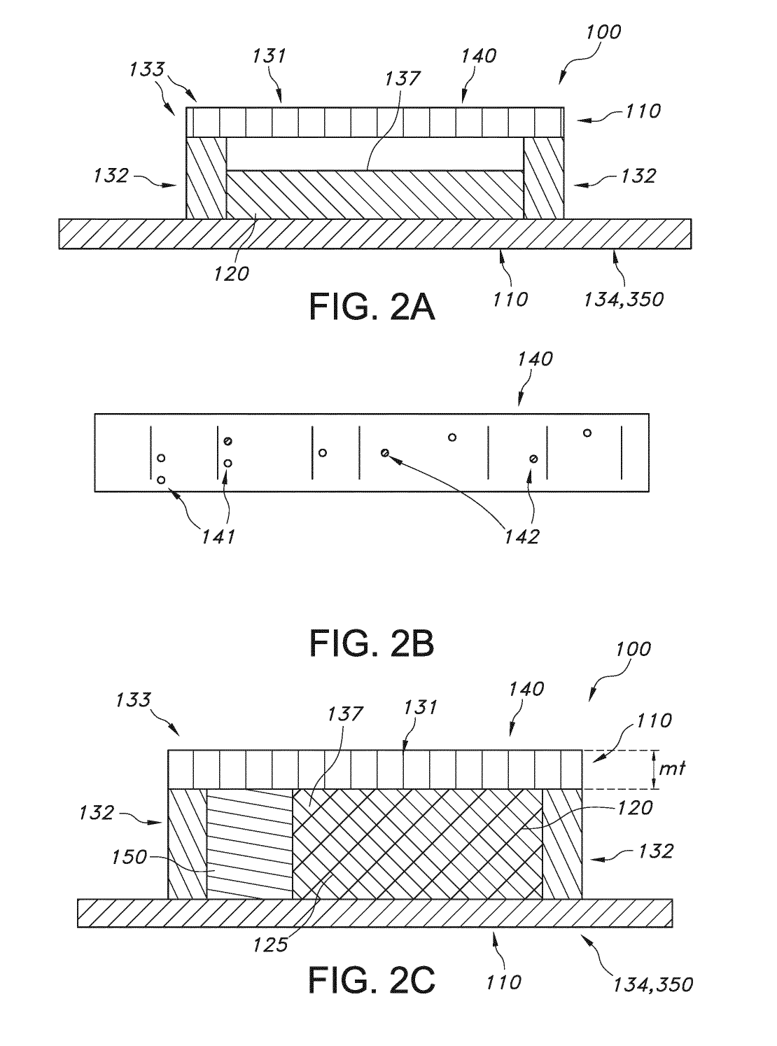

FIGS. 2a-2c schematically depict some embodiments of the CO₂ detector, which is indicated with reference 100. The CO₂ detector 100 is thus especially configured for application in a closed space 1 enclosing a liquid (food), see also FIG. 1a. The CO₂ detector 100 comprises a detector enclosure 110 containing a pH indicator 120. The detector enclosure 110 is impermeable to liquid water. Especially, at least a first part 131 of the detector enclosure 110 comprises a silicone membrane 140 that is permeable for CO₂. One or more of the first part 131 and an optional second part 132 of the detector enclosure 110 comprises a light transmissive part 133 for external visual inspection of the pH indicator 120 of the CO₂ detector 100. For instance, the silicone material may be transmissive for light, and can thus be used for inspection of the pH indicator 120. FIG. 2b schematically shows an embodiment wherein the silicone membrane 140 comprises one or more of a UV absorber material 141 and a UV reflector material 142. FIG. 2c schematically depicts an embodiment wherein the CO₂ detector 100 further comprises a CO₂-detector end-of-life indicator 150. Also such end-of-life indicator may be based on a color change, and may in embodiments be configured behind a light transmissive part of the detector enclosure. By way of example, in FIG. 2c the detector enclosure 110 where the pH indicator 120 is configured also includes water or another aqueous liquid, indicated with reference 125. Reference 134 indicates a substrate. Here, at least part of the substrate 134 is configured as detector enclosure 110. Reference 137 indicates the space enclosed by the detector enclosure 110, which here contains at least the pH indicator 120. This space may e.g. have a volume of about 0.1-10,000 mm³. Reference mt in FIG. 2c indicates the membrane thickness.

Referring to e.g. FIGS. 2a and 2c, the detector enclosure 110 may be the support 350 or at least part of the detector enclosure may be comprised by the support. The latter embodiment is schematically depicted in FIGS. 2a and 2c.

Referring to e.g. FIGS. 2a and 2c, the detector enclosure and space are different parts.

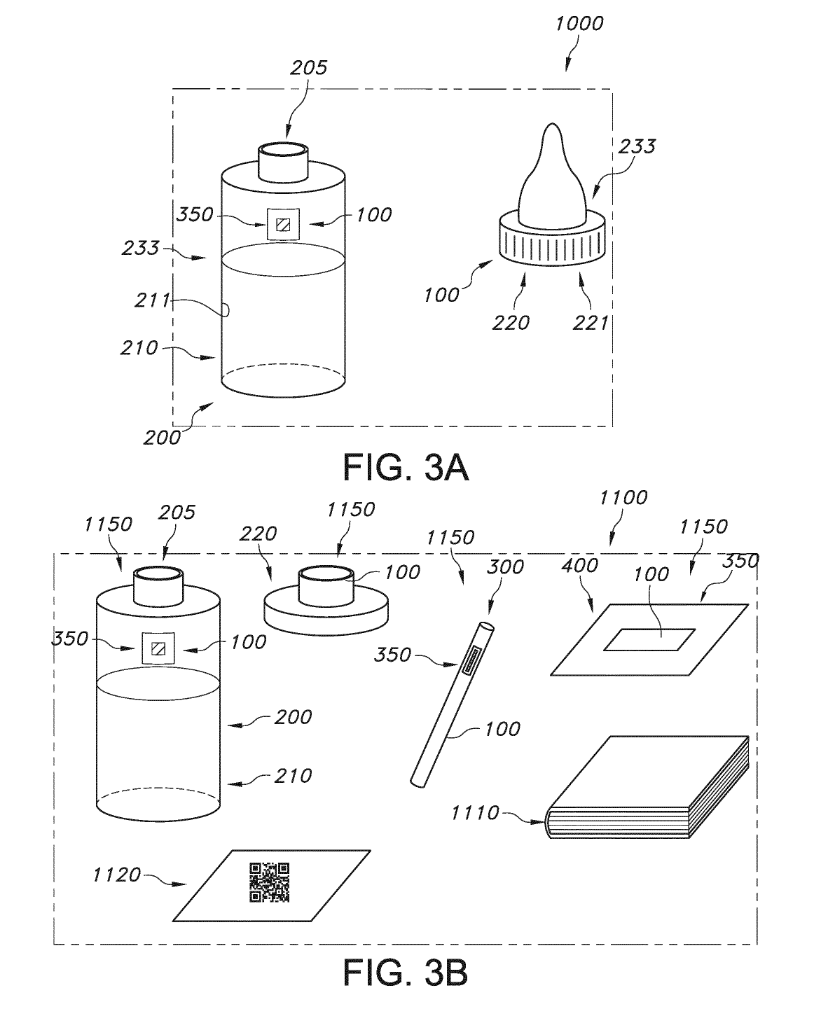

FIGS. 3a-3b schematically depict some embodiments of kits of parts. FIG. 3a schematically depicts a kit of parts 1000 comprising a container 200; here in this schematic drawing, the container 200 is not closed, but opened. As indicated above, the container 200 comprises a container enclosure 210 comprising a container enclosure internal face 211. Further, the kit of parts 1000 comprises a container closure 220. The container closure 220 comprises a container closure internal face 221. One or more of the container enclosure internal face 211 and the container closure internal face 221 may comprise the CO₂ detector 100. Here, by way of example both are indicated to include such CO₂ detector, though this may not be necessary. Note that the CO₂ detector 100 comprised by the container closure 220 is configured within the container closure 220 and is thus not visible in this schematic drawing (see however e.g. FIG. 1b ). Further, the kit may comprise a plurality of container enclosures 210 and/or a plurality of container closures 220. Further, one or more of the container enclosure 210 and the container closure 220 comprise a light transmissive part 233 for external visual inspection of the CO₂ detector 100. Reference 205 indicates the opening in the container enclosure 210 which is closable with the container closure 220 (for providing a closed container 200).

FIG. 3b schematically depicts another kit of parts 1100, which comprises one or more items 1150 selected from the group consisting of the container enclosure 210, the container closure 220, the elongated positioning element 300, and the reusable sticker 400. One or more of these items 1150 comprise the CO₂ detector 100. Further, the kit of parts 1100 may comprise one or more of a manual 1110 or a link 1120 to a manual on the internet. Here, by way of example both a manual 1110 and the link 1120 are schematically depicted.

The term “kit of parts” is herein used to indicate a plurality of (different) items that e.g. may be functionally used together.

Elements of the invention may include a pH indicator (e.g., malachite green, phenol red, thymol blue, etc.), a carrier (e.g. filter paper), a silicone shield. Further, optionally a liquid or aqueous solution to help promote dissolution of carbon dioxide for faster response time of the pH indicator may be applied. An adhesive may be applied to enable attachment to a lid or wall of a baby bottle or milk cup, especially a food safe adhesive (e.g., Fasson® AT1, Permabond ET5145, etc.). Further, optionally a second pH indicator, such as a sticker with pH indicator, can be used to show that the kit of parts is still operational and did not deteriorate.

Amongst others, an aspect of the proposed invention may be to employ e.g. a 1-5 mm thick semi-porous, hydrophobic and biocompatible silicone layer to shield a pH indicator (e.g., malachite green, phenol red, thymol blue, etc.), such as immobilized on a carrier (e.g., filter paper), to prevent leaching of pH indicator into the milk. In addition, the in situ pH measurement is enabled by diffusion of gaseous carbon dioxide (produced as a by-product of the milk spoilage/fermentation process) in the air above the milk or in the milk itself through the silicone shield until it comes into contact with the pH indicator. The pH indicator will change color as a result of changes in the carbon dioxide concentration in the milk or in the air above it, as well as all other volatile acidic and basic (i.e., alkaline) gases. As the milk becomes acidic, the alkaline substances become less important. In addition to CO₂, the other main acidic compound in the milk is acetic acid. This visual color change is used to indicate to the user the spoilage state of the milk.

To enhance the response time of the pH indicator a small volume (1-5 ml) of liquid (e.g., water) or an aqueous solution may be added to the enclosure containing the pH indicator will promote faster reaction of the pH indicator with the gaseous carbon dioxide. In the case of water, dissolution of the carbon dioxide (CO₂) leads to the formation of carbonic acid (H₂CO₃) which will then react with pH indicator:

CO₂(g)<↔CO₂(aq.) (1)

CO₂(aq.)+H₂O(1)<↔H₂CO₃(aq.) (2)

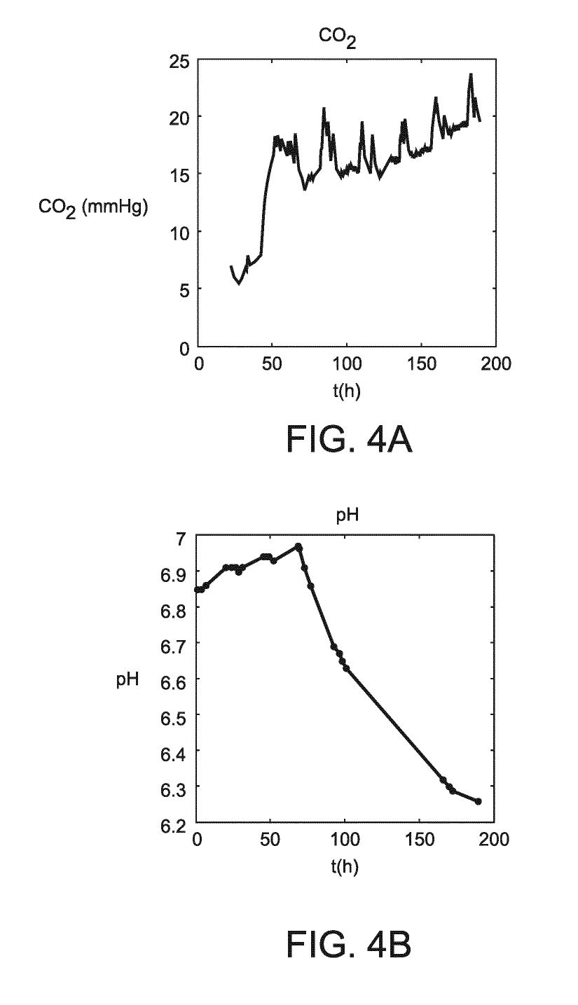

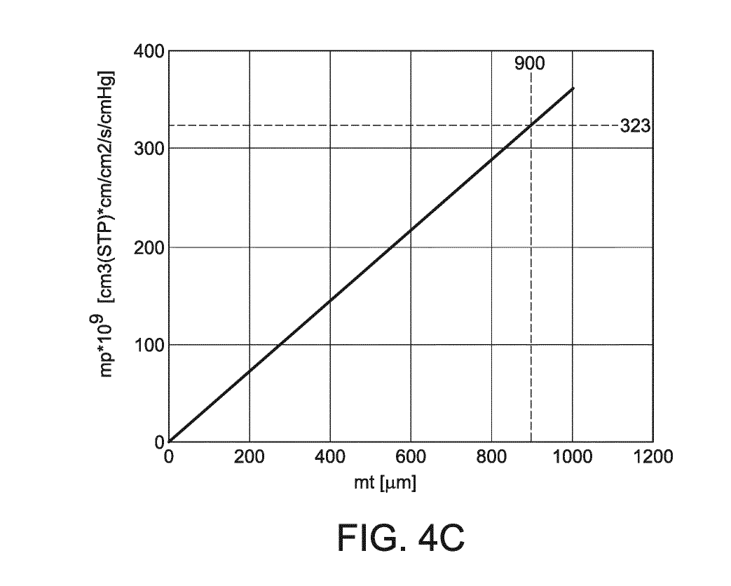

Milk age—Older milk will produce more carbon dioxide than fresher milk because the fermentation process is further advanced. As milk spoils its pH decreases from approximately neutral (pH≈7) to acidic (pH=5.0-6.5), which is associated with an increase in carbon dioxide from very low concentrations (CO₂ partial pressure=2.39 mmHg) in fresh milk to higher concentrations (CO₂ partial pressure=71.54 mmHg) in spoiled milk. This can also be seen in FIGS. 4a-4b during the transition of the milk from a fresh to spoiled state over a period of several days. In this case a four-fold increase in CO₂ partial pressure is observed from z5mmHg up to z23.5mmHg, with a corresponding decrease in pH from 6.85 to 6.26. It is important that this is independent of milk volume since the CO₂ concentration in the gas phase depends on CO₂ concentration in the milk, the pH of the milk, the temperature of the milk, the kind of milk and milk composition. Although smaller volumes contains less CO₂, however, the concentration in partial pressure is the same for small and big volumes provided enough CO₂ is present. In FIG. 4a, at the steep increase at about 50 hours, the milk starts to spoil. A sharp change in pH of the milk is only at about 75 hours. Hence, a CO₂ detector 100 in the headspace may be a better indicator than a pH indicator configured within the milk.

If the proposed invention is placed in a baby bottle, milk pouch or milk cup with fresh (unspoiled) breast or formula milk, the small amount of carbon dioxide that is present will react with the pH indicator to produce a neutral pH reading (i.e., pH=6.8-7.4), resulting in a visual color change which indicates the milk is not spoiled. This color change will also help the user to know when the measurement is done (e.g., in case milk is poured in from another container). In contrast if spoiled (sour) breast or formula milk is used, the large amount of carbon dioxide present will react with the pH indicator to produce an acidic pH reading (pH<6.8) and a corresponding color change. The response time of the spoilage indicator will be on the order of≈10-30 s.

Moreover, the proposed milk spoilage solution is reusable and serializable since the color change of the pH indicator is reversible. The carbon dioxide that reacts with the pH indicator can be displaced by air or steam thereby reversing the reaction. It is also washable/cleanable since it is made from silicone which is hydrophobic and will not swell or absorb any liquid from the milk. It is expected that the mother can use the indicator for several dozen times (potentially up to 100 times) before she has to replace it.

FIG. 4c depicts the membrane permeability versus thickness of the membrane, assuming a reaction time of 5 minutes (low CO₂ to high CO₂ concentration), at room temperature. The graph shows the membrane thickness required for a material of a certain CO₂ permeability. The case of silicone is highlighted, with a permeability of 323*10⁹ cm³ (STP) cm cm⁻² s⁻¹ cmHg⁻¹. The maximum thickness for 5 minutes reaction time is 900 um. This graph shows, that if a material (any material) with a lower permeability for CO₂ than silicone is used, the membrane (=shielding material) thickness should decrease linear, in order to obtain the same reaction time for the sensor.

The term “substantially” herein, such as in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices herein are amongst others described during operation. As will be clear to the person skilled in the art, the invention is not limited to methods of operation or devices in operation.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

The invention further applies to a device comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A CO₂ detector for application in a closed space enclosing a liquid food, wherein the CO₂ detector comprises a detector enclosure containing a pH indicator, wherein the detector enclosure is impermeable to liquid water, wherein at least a first part of the detector enclosure comprises a silicone membrane that is permeable for CO₂, wherein one or more of the first part and an optional second part of the detector enclosure comprises a light transmissive part for external visual inspection of the pH indicator of the CO₂ detector.

2. The CO₂ detector according to claim 1, wherein the silicone membrane is permeable to water vapor, wherein the permeability for water vapor is at least 20,000 cm³ (STP) cm cm⁻² s⁻¹ cmHg⁻¹, wherein the silicone membrane is light transmissive, wherein the silicone membrane is hydrophobic, and wherein the permeability for CO₂ is at least 1,000 cm³ (STP) cm cm⁻² s⁻¹ cmHg⁻¹.

3. The CO₂ detector according to claim 1, wherein the detector enclosure has a volume in the range of 0.1-10,000 mm³, and wherein the detector enclosure also contains water.

4. The CO₂ detector according to claim 1, wherein the pH indicator is configured to change color at a pH selected from the range of 5-7.

5. The CO₂ detector according to claim 1, wherein the pH indicator comprises a pH indicator ion bound to a polymeric ion.

6. The CO₂ detector according to claim 1, wherein silicone membrane comprises polydimethyl siloxane.

7. The CO₂ detector according to claim 1, wherein the silicone membrane comprises one or more of a UV absorber material and a UV reflector material, wherein the CO₂ detector further comprises a CO₂-detector end-of-life indicator, and wherein the membrane thickness (μm) is selected from 900 μm*323.10⁹ cm³ (STP) cm cm⁻² s⁻¹ cmHg⁻¹/(P)+/−50%, wherein the thickness is larger than 0 μm, and wherein P is the permeability in 10⁹ cm³(STP) cm cm⁻² s⁻¹ cmHg₋₁ of a material of the membrane.

8. A kit of parts comprising a container comprising a container enclosure, the container enclosure comprising a container enclosure internal face, and a container closure, the container closure comprising a container closure internal face, wherein one or more of the container enclosure internal face and the container closure internal face comprise the CO₂ detector according to claim 1, and wherein one or more of the container enclosure and the container closure comprise a light transmissive part for external visual inspection of the CO₂ detector.

9. The kit of parts according to claim 8, wherein the container closure internal face comprises said CO₂ detector.

10. The kit of parts according to claim 8, wherein the CO₂ detector is detachable associated with the container enclosure internal face or the container closure internal face.

11. The kit of parts according to claim 8, wherein the container is configured to contain expressed breast milk.

12. An elongated positioning element, configured to position in a container, wherein the positioning element comprises the CO₂ detector according to claim 1.

13. A reusable sticker comprising the CO₂ detector according to claim 1.

14. A kit of parts comprising (a) one or more items selected from the group consisting of the container enclosure, the elongated positioning element, and the reusable sticker, wherein one or more items comprise the CO₂ detector according to claim 1, and wherein the kit of parts further comprises (b) one or more of a manual or a link to a manual on the internet.

15. A method of evaluating expressed breast milk, the method comprising inspecting a CO₂ detector according to claim 1 in a container or retrieved from the container, wherein the container contains expressed breast milk, and wherein inspection is executed after the CO₂ detector has been contained in the container in a headspace over said expressed breast milk during a period wherein the container is closed with a container closure.
